(19) United States
(12) Patent Application Publication
(10) Pub. No.: US 2024/0315662 A1
(43) Pub. Date: Sep. 26, 2024

(54) Telemedicine Stethoscope for Enhanced Remote Auscultation

(71) Applicants: Haluk Un, WOODINVILLE, WA (US); MUSTAFA YASAR SIR, WOODINVILLE, WA (US)

(72) Inventors: Haluk Un, WOODINVILLE, WA (US); MUSTAFA YASAR SIR, WOODINVILLE, WA (US)

(21) Appl. No.: 18/609,057

(22) Filed: Mar. 19, 2024

Related U.S. Application Data
(60) Provisional application No. 63/453,336, filed on Mar. 20, 2023.

(51) Int. Cl. A61B 7/04; A61B 5/00; H04R 1/46

(52) U.S. Cl. CPC A61B 7/04; A61B 5/6898; A61B 5/7465; H04R 1/46; A61B 2560/0443; A61B 2562/0204

(57) ABSTRACT

The present invention relates to a telemedicine stethoscope device that facilitates remote medical consultations by enabling the transmission of bodily sounds from a patient to a healthcare provider through an electronic device. The device consists of a skin contact element designed to establish a secure interface with the patient's skin, a walled structure that connects the skin contact element to a base, and a detachable coupling mechanism incorporated into the base for removably attaching the device to an electronic device. A diaphragm membrane, made of a flexible material and positioned orthogonally within the path between two openings, forms a sound chamber with the hollow space to effectively transmit sound vibrations from the patient's skin through the device to the microphone of the electronic device.

### FIELD OF INVENTION

The present invention relates generally to the field of medical devices, specifically to an advanced telemedicine stethoscope designed for remote auscultation, enabling the recording and transmission of heart, lung, and bowel sounds during virtual consultations.

### BACKGROUND

Telemedicine, a critical component of modern healthcare delivery, has enabled remote patient consultations, providing significant benefits in terms of accessibility and convenience. Despite these advancements, a considerable challenge remains in replicating the depth and quality of physical examinations conducted in-person, particularly in the context of auscultation. This limitation hinders physicians' ability to listen to crucial bodily sounds such as those from the heart, lungs, and intestines during video conference consultations.

The existing solutions, which include a variety of electronic stethoscopes and sound transmission devices, have sought to address this gap. However, they have fallen short due to several significant drawbacks, including high costs, complex operation, and suboptimal sound quality, which limit their practical utility, especially for patients without medical training.

These devices often necessitate the acquisition of additional electronic hardware, escalating costs and complicating maintenance and operation. For instance, electronic stethoscopes, which record and transmit sound to a communication device, not only increase the financial burden but also require users to manage device charging and maintenance, further complicating their use. Additionally, the design of these devices frequently does not accommodate the anatomical and physiological diversity of the patient population. Devices equipped with large diaphragms, intended to enhance sound collection, struggle with maintaining optimal skin contact across all patients, particularly those with lower body fat or pediatric patients, resulting in poor sound quality. This issue is exacerbated in the thoracic region, where the presence of rib bones and potential gaps can significantly diminish the efficacy of sound transmission.

Moreover, the interface between the stethoscope and the electronic device, crucial for sound transmission, is often flawed. Designs that fail to ensure airtight contact between the microphone and the stethoscope lead to compromised sound quality due to air gaps or inadequate sealing. Such shortcomings underscore the necessity for a novel approach that not only overcomes these limitations but is also affordable, accessible, and compatible with a wide array of electronic devices. The proposed invention seeks to address these challenges by introducing a stethoscope designed for telemedicine that ensures effective sound transmission while being user-friendly and versatile, capable of attaching to various electronic devices without the need for additional hardware. This approach not only aims to enhance the quality of remote physical examinations but also to democratize access to comprehensive healthcare consultations, irrespective of geographical limitations or socioeconomic barriers. The design considerations also extend to optimizing user interaction with the device, incorporating clear instructions and the potential integration of AI models to further improve the usability and effectiveness of the stethoscope in telemedicine applications.

Several prior art disclosures in the realm of telemedicine and acoustic collection systems for electronic devices reveal a consistent set of deficiencies that limit their utility and adaptability in clinical and remote medical settings.

Notably, patent applications such as U.S. Ser. No. 10/898,161B2 describe acoustic collection systems that are hindered by a broad diaphragm base, making them unsuitable for placement in the intercostal spaces where precise sound collection is often required. This structural limitation restricts their effectiveness in accurately capturing critical heart and lung sounds, a fundamental aspect of patient assessment.

Additionally, the complexity of the designs detailed in patent applications like U.S. Pat. No. 9,474,489B2 and U.S. Pat. No. 9,602,917B2 not only escalates production costs but also results in a lack of adaptability to various models and sizes of handheld electronic devices. This rigidity diminishes the practicality of these inventions, as it fails to accommodate the diverse range of devices used in telehealth consultations today.

Further, the system and method outlined in US20150087926A1, along with the telephone-based apparatus in US20220068505A1, share these fundamental drawbacks. Their broad diaphragm bases preclude effective use in anatomically narrow areas, and their intricate designs contribute to high manufacturing expenses and a fixed compatibility range, limiting their widespread application.

International and regional patent applications such as WO2021163135A1 and EP3242600B1, along with KR20130041559A and KR101511099B1, also exhibit these critical limitations. They feature broad diaphragm bases that are not conducive to intercostal auscultation and display a lack of versatility in adapting to different electronic devices, thereby constraining their usability across various telemedicine platforms.

The common thread among these patent applications is a design that does not fully address the practical needs of remote medical diagnostics and consultations. Their inability to snugly fit into the intercostal spaces for optimal sound collection, coupled with high production costs and a lack of adaptability to a broad spectrum of electronic devices, underscores the need for an improved telemedicine stethoscope device. Such an invention would ideally overcome these deficiencies, offering a more flexible, cost-effective, and universally compatible solution for enhancing remote patient assessments.

It is within this context that the present invention is provided.

### SUMMARY

The present invention relates to a telemedicine stethoscope device designed to improve remote medical consultations by enabling the transmission of heart, lung, and bowel sounds from a patient to a healthcare provider through an electronic device. The device comprises a skin contact element with a first opening for secure interface with the patient's skin, a walled structure connecting the skin contact element to a base with a second opening, a detachable coupling mechanism for attaching the device to an electronic device, and a diaphragm membrane that works with the hollow space to form a sound chamber for transmitting sound vibrations effectively.

In some embodiments, the skin contact element is enhanced with an elastic and soft material that adapts to various skin surfaces. This material's ability to temporarily adhere to the skin ensures that the device remains stationary during operation, thereby maintaining the quality of sound transmission without interference from movement.

In other embodiments, the detachable coupling mechanism includes an adhesive layer, offering a secure yet easily removable attachment to various electronic devices. This flexibility allows for quick setup and teardown, facilitating seamless integration into telemedicine consultations.

Further embodiments include a casing as part of the detachable coupling mechanism. This casing encapsulates the electronic device, creating a sealed environment that enhances sound transmission directly from the stethoscope to the electronic device's microphone, thereby improving sound quality and reducing ambient noise.

Additionally, some embodiments feature protrusions on the detachable coupling mechanism designed for alignment with specific features on an electronic device, such as a charging port or audio jack. These protrusions ensure precise placement of the stethoscope relative to the electronic device's microphone, optimizing sound capture.

The diaphragm membrane in certain embodiments is either transparent or semi-transparent, allowing visual inspection of its positioning and condition, whereas other embodiments utilize an opaque diaphragm membrane, focusing solely on sound transmission without visual distraction.

The walled structure of the stethoscope may include side walls that taper inward or outward. This design optimizes the acoustic properties of the sound chamber, enhancing the fidelity of sound transmission from the patient to the healthcare provider.

Constructed from a variety of materials such as plastic, rubber, silicone, metal, or fabric, the device offers durability and flexibility, catering to diverse manufacturing and usage requirements.

Some embodiments feature a protective cover over the base, which may contain adhesive properties for secure placement on the electronic device, along with openings to accommodate alignment protrusions or other features, ensuring the device's stability during use.

The device is further characterized by multiple microphone openings located on the base, accommodating different models of electronic devices. This feature ensures universal compatibility and efficient sound transmission across various types of electronic devices.

In embodiments where the hollow space between the first and second openings is adjustable, the device can modify the acoustics of the sound chamber to accommodate variations in patient anatomy and specific auscultation requirements, providing tailored diagnostic capabilities.

Designed for integration with or attachment to a variety of electronic devices, including smartphones, tablets, and wearable technology, the device enables versatile use in telemedicine applications, broadening its applicability across different healthcare settings.

Incorporating artificial intelligence models, the device optimizes audio-quality assessment for the detection and analysis of heart, lung, and gastrointestinal sounds. This technology enhances the device's diagnostic capabilities, offering healthcare providers a more nuanced and accurate understanding of a patient's condition remotely.

### BRIEF DESCRIPTION OF THE DRAWINGS

Various embodiments of the invention are disclosed in the following detailed description and accompanying drawings.

FIG. 1 is a perspective view an example configuration of the parts that make up the stethoscope.

FIG. 2A is a perspective view of an example configuration the stethoscope.

FIG. 2B is a top view of an example configuration the stethoscope.

FIG. 2C is a bottom view of an example configuration the stethoscope.

FIG. 3A is a side-sectional view of an example configuration the stethoscope.

FIG. 3B is a side-sectional view of an example configuration the stethoscope.

FIG. 3C is a side-sectional view of an example configuration the stethoscope.

FIG. 4A is a side-sectional view of an example configuration the stethoscope.

FIG. 4B is a perspective view of an example configuration the stethoscope.

FIG. 4C is a perspective view of an example configuration the stethoscope.

FIG. 5A is a perspective view of an example configuration the electronic device case folded with the stethoscope.

FIG. 5B is a perspective view of an example configuration the electronic device case with the stethoscope.

FIG. 6A is a perspective view of an example configuration the electronic device case and stethoscope on the electronic device.

FIG. 6B is a side view of an example configuration the electronic device case and stethoscope on the electronic device.

FIG. 6C is a bottom view of an example configuration the electronic device case and stethoscope on the electronic device.

FIG. 7A is a side view of an example configuration the stethoscope.

FIG. 7B is a side view of an example configuration the stethoscope.

FIG. 7C is a side-sectional view of an example configuration the stethoscope.

FIG. 8A is a bottom view of an example configuration the stethoscope.

FIG. 8B is a top view of an example configuration the stethoscope.

FIG. 8C is a side view of an example configuration the stethoscope.

FIGS. 9A, 9B, and 9C are side-sectional views of an example configuration the stethoscope.

FIG. 10A is a bottom view of an example configuration the stethoscope.

FIG. 10B is a top view of an example configuration the stethoscope.

FIGS. 11A and 11B are side-sectional views of an example configuration the stethoscope.

FIG. 12A is a side-sectional view of an example configuration the stethoscope.

FIG. 12B is a top view of an example configuration the stethoscope.

FIG. 13A is a side view of an example configuration the stethoscope.

FIG. 13B is a front view of an example configuration the stethoscope.

FIG. 14 is a front view of an example configuration the electronic device case with the stethoscope.

FIGS. 15A, 15B, 15C, and 15D are side-sectional views an example configuration of the stethoscope.

Common reference numerals are used throughout the figures and the detailed description to indicate like elements. One skilled in the art will readily recognize that the above figures are examples and that other architectures, modes of operation, orders of operation, and elements/functions can be provided and implemented without departing from the characteristics and features of the invention, as set forth in the claims.

### DETAILED DESCRIPTION AND PREFERRED EMBODIMENT

The following is a detailed description of exemplary embodiments to illustrate the principles of the invention. The embodiments are provided to illustrate aspects of the invention, but the invention is not limited to any embodiment. The scope of the invention encompasses numerous alternatives, modifications and equivalent; it is limited only by the claims.

Numerous specific details are set forth in the following description in order to provide a thorough understanding of the invention. However, the invention may be practiced according to the claims without some or all of these specific details. For the purpose of clarity, technical material that is known in the technical fields related to the invention has not been described in detail so that the invention is not unnecessarily obscured.

### Definitions

The terminology used herein is for the purpose of describing particular embodiments only and is not intended to be limiting of the invention.

As used herein, the term “and/or” includes any combinations of one or more of the associated listed items.

As used herein, the singular forms “a,” “an,” and “the” are intended to include the plural forms as well as the singular forms, unless the context clearly indicates otherwise.

It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

When a feature or element is described as being “on” or “directly on” another feature or element, there may or may not be intervening features or elements present. Similarly, when a feature or element is described as being “connected,” “attached,” or “coupled” to another feature or element, there may or may not be intervening features or elements present. The features and elements described with respect to one embodiment can be applied to other embodiments.

The use of spatial terms, such as “under,” “below,” “lower,” “over,” “upper,” etc., is used for ease of explanation to describe the relationship between elements when the apparatus is in its proper orientation.

The terms “first,” “second,” and the like are used to distinguish different elements or features, but these elements or features should not be limited by these terms. A first element or feature described can be referred to as a second element or feature and vice versa without departing from the teachings of the present disclosure.

As used herein, the term “telemedicine stethoscope device” refers to an apparatus designed to enable the recording and transmission of bodily sounds, such as heart, lung, and bowel sounds, from a patient to a healthcare provider through an electronic device during remote medical consultations. This encompasses devices that are capable of attaching to electronic devices, such as smartphones or tablets, to facilitate auscultation in a telemedicine setting.

For the purposes of this disclosure, the term “skin contact element” is understood to mean any part or component of the telemedicine stethoscope device that is intended to make direct contact with the patient's skin for the purpose of sound transmission. This element may include, but is not limited to, surfaces made of soft, pliable materials or those with adhesive properties to enhance contact stability and sound quality.

The term “detachable coupling mechanism” as used herein encompasses any means or method that allows the telemedicine stethoscope device to be securely attached to and easily detached from an electronic device. Examples of such mechanisms include, but are not limited to, adhesive layers, mechanical clamps, magnetic attachments, or cases specifically designed to hold both the stethoscope device and the electronic device.

As mentioned, the “diaphragm membrane” is a key component of the telemedicine stethoscope device, constructed from a flexible material and positioned to facilitate the effective transmission of sound vibrations. The term includes membranes made of materials such as silicone, rubber, or any suitable polymer that can vibrate in response to sound waves, thereby converting acoustic energy into mechanical vibrations that can be transmitted to the electronic device.

The phrase “sound chamber,” as utilized herein, refers to the space or cavity formed between the skin contact element and the microphone of the electronic device, bound by the diaphragm membrane and the internal surfaces of the device. This chamber is crucial for the amplification and clarity of the sound signals being transmitted. The design of the sound chamber, including its shape and volume, can vary to optimize sound quality for different types of bodily sounds.

### DESCRIPTION OF DRAWINGS

The present disclosure pertains to a telemedicine stethoscope device, a novel apparatus designed to facilitate the remote medical examination of patients by enabling the transmission of bodily sounds to healthcare providers through an electronic device. This invention addresses the need for effective remote auscultation capabilities within the context of telemedicine, offering a practical solution for healthcare professionals to conduct comprehensive assessments of heart, lung, and bowel sounds during video conference consultations.

The telemedicine stethoscope device is comprised of a skin contact element, a diaphragm membrane, a hollow body, and a detachable coupling mechanism. The skin contact element, made from a soft, thin, potentially elastic, and sticky material, ensures a stable and secure interface with the patient's skin, minimizing movement and noise interference. This element features a first opening integral to sound acquisition, leading to a diaphragm membrane—a flexible, possibly transparent or semi-transparent thin layer that is key for collecting sounds. The body of the stethoscope, which can be either single or multi-layered and designed with symmetrical or asymmetrical surfaces, encloses a hollow space extending from the first to a second opening. This space forms a sound chamber in conjunction with the diaphragm membrane, optimizing sound vibration transmission from the skin to the device. Incorporated into the base of the stethoscope is a detachable coupling mechanism that enables the device to be attached to an electronic device securely, aligning the second opening with the electronic device's microphone for sound transfer. This connection can be achieved through adhesive layers or specialized cases, which are adaptable and designed to create a tight seal around the microphone, ensuring clear sound transmission.

The stethoscope and its components can be constructed from a variety of materials such as plastic, wood, and silicone, chosen for their flexibility and suitability for medical applications. These materials, combined with the device's innovative design, allow for easy integration with electronic devices, facilitating effective telemedicine practices.

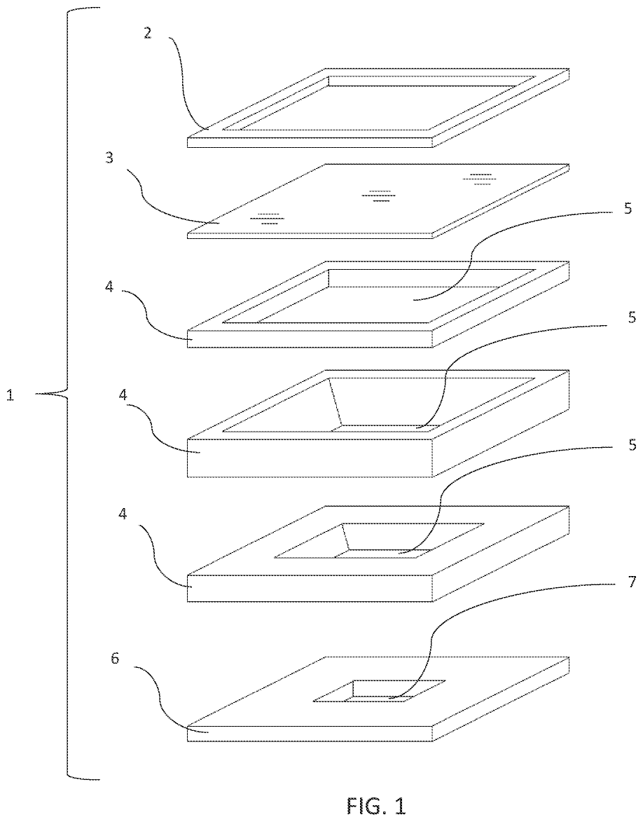

FIG. 1 presents a perspective view of the telemedicine stethoscope device (1), showcasing its various components in sequential and separated positions for clarity. At the forefront of the device is the skin grip area (2), designed to be the initial point of contact with the patient's skin, encircling the diaphragm (3). This crucial area (2) is fabricated from a soft, pliable material, meticulously chosen to be thin enough to ensure an unobstructed connection with the skin while providing sufficient grip to prevent slippage. Located directly beneath this area is the diaphragm (3), a thin membrane responsible for the efficient transmission of sound from the external environment to the internal mechanism of the stethoscope. The diaphragm (3) is carefully integrated within the structure, bordered by the skin grip area (2) to optimize sound collection.

The body of the stethoscope (4) is depicted as a hollow, box-shaped structure, housing a central void (5) that serves as the conduit for sound waves captured by the diaphragm (3). This body (4) can be constructed from either a single piece or multiple interconnected parts, contributing to the device's modular design. This arrangement facilitates sound's progression from the diaphragm (3) through to the microphone opening (7) located on the base (6) of the device. The base (6) itself is engineered to support attachment mechanisms for securing the stethoscope (1) to various electronic devices (14) or protective covers (8), with the microphone opening (7) precisely positioned to align with and capture audio from the electronic device's microphone (16).

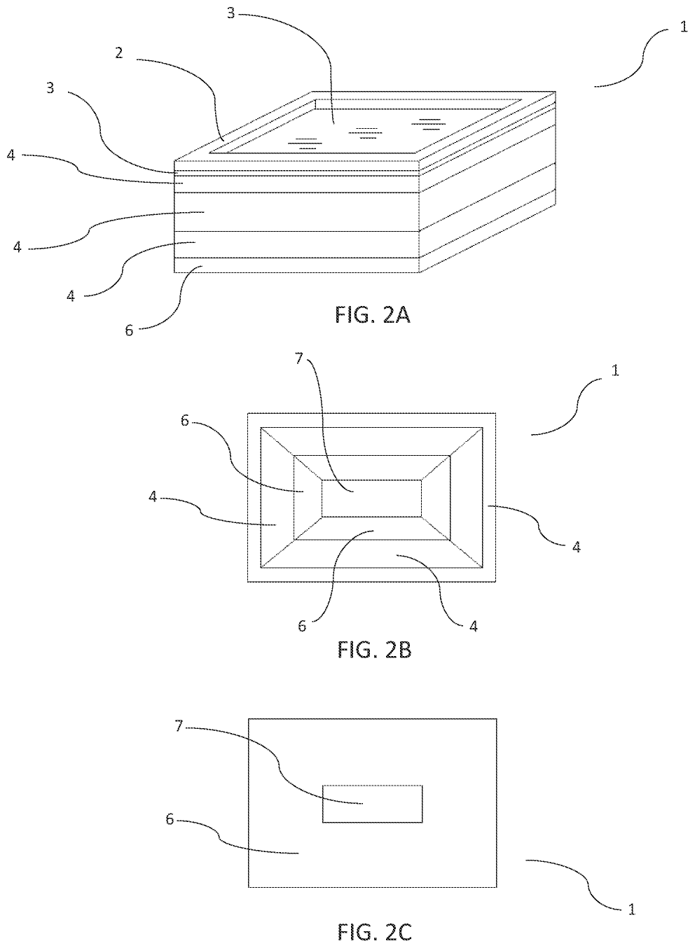

FIG. 2A provides a comprehensive perspective view of the assembled stethoscope device (1), highlighting its cohesive structure. The skin grip area (2) is positioned prominently at the top, seamlessly transitioning to the diaphragm (3) and subsequently to the main body (4) of the stethoscope, emphasizing its hollow, box-like form.

In FIG. 2B, the top view of the stethoscope (1) is revealed, illustrating the transition from the broader upper section to the narrower lower sections. This design consideration ensures that the microphone opening (7) at the base (6) is optimally located to interface with the microphone (16) of the electronic device (14), thereby enhancing sound transfer efficacy.

FIG. 2C's bottom view of the stethoscope (1) displays the strategic placement of one or more openings (7) within the base (6). These openings (7) are crucial for the direct transmission of sound waves from the diaphragm (3) to the electronic device's microphone (16). Their design varies to include circular, polygonal, elliptical, or irregular shapes.

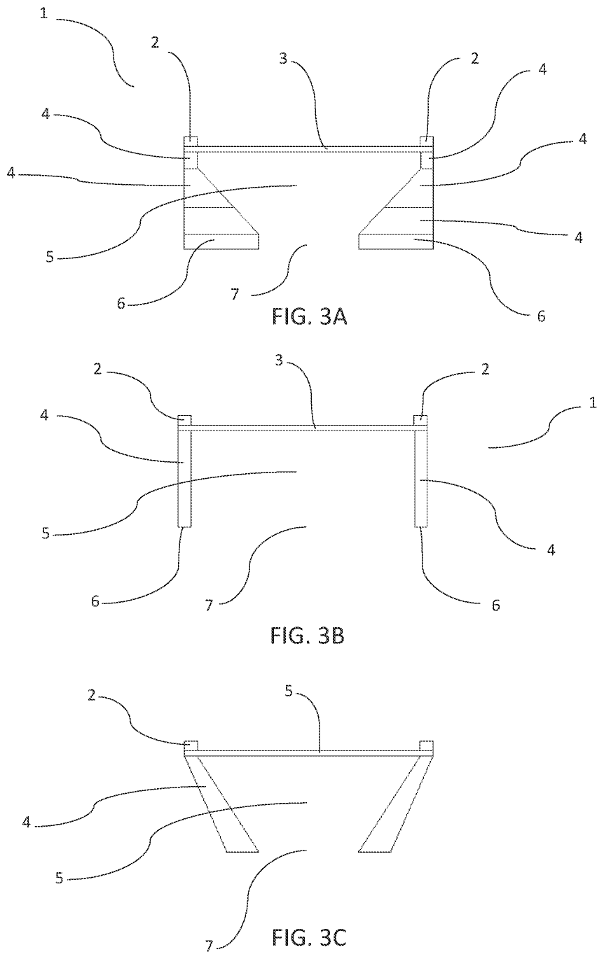

FIG. 3A offers a side cross-sectional view of the telemedicine stethoscope device (1), revealing the variability in the shape and structure of its body (4). At the pinnacle of this assembly is the skin grip area (2), meticulously designed to ensure optimal contact with the patient's skin. Directly beneath this, the diaphragm (3) spans the entirety of the upper section, with potential for a minute opening aimed at modulating air pressure within the device. The composition of the body (4) beneath the diaphragm (3) is characterized by varying thicknesses and can exhibit either a symmetrical or asymmetrical form.

In the depiction provided by FIG. 3B, an alternative cross-sectional perspective showcases the stethoscope's body (4) as potentially symmetric and uniformly flat. This variant, which could be crafted as a singular piece or from a conglomerate of multiple elements, demonstrates the versatility in design. The parallelism between the microphone opening (7) and the central void (5) highlights the precision in sound channeling, with the possibility of these apertures varying in width to accommodate different acoustic profiles.

FIG. 3C illustrates yet another cross-sectional view where the body (4) tapers towards the base (6) and microphone opening (7), with the sides slanting inward. This design ensures that the diaphragm (3) is afforded a wider expanse at the point of skin contact, while the base (6) remains narrower, facilitating a focused and efficient sound transmission pathway.

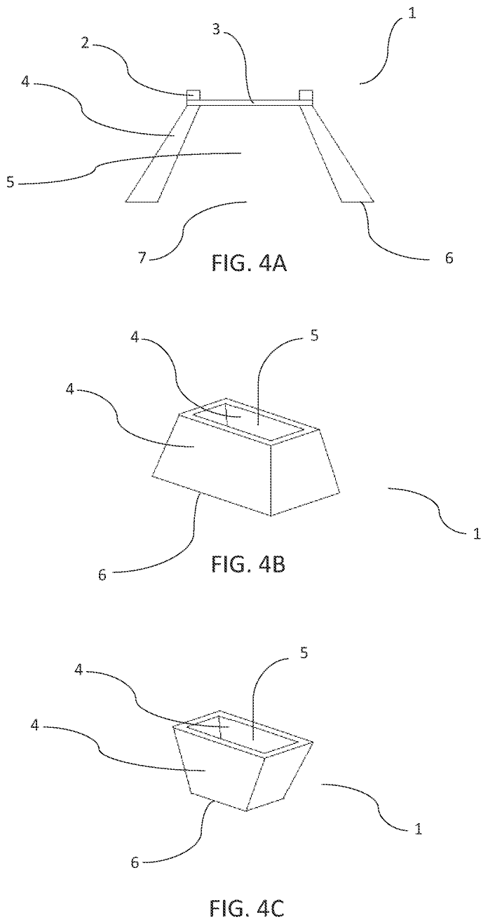

Similarly, FIG. 4A presents an alternative design where the side walls of the body (4) taper outward as they approach the base (6) and microphone opening (7). This configuration inversely narrows the diaphragm (3) region compared to the base (6), optimizing the device for specific anatomical and acoustic considerations. In each of these configurations, the body (4) and the base (6) are integrated as single units.

FIG. 4B offers a perspective view of the telemedicine stethoscope device (1), specifically illustrating the design variant from FIG. 4A. In this rendition, the diaphragm (3) portion of the stethoscope (1) is deliberately designed to be narrower in comparison to the broader base (6), enhancing the device's adaptability to various patient anatomies. Both the body (4) and the base (6) of the stethoscope are manufactured as a single, cohesive unit, streamlining the device for ease of use and maintenance.

FIG. 4C provides another perspective view, this time of the stethoscope (1) configuration as seen in FIG. 3C. Here, the design emphasizes a wider diaphragm (3) section relative to a more slender base (6), optimizing the sound collection surface area for enhanced acoustic sensitivity. This design, too, features the body (4) and the base (6) as integrated components, ensuring structural integrity and functional consistency across various operational contexts.

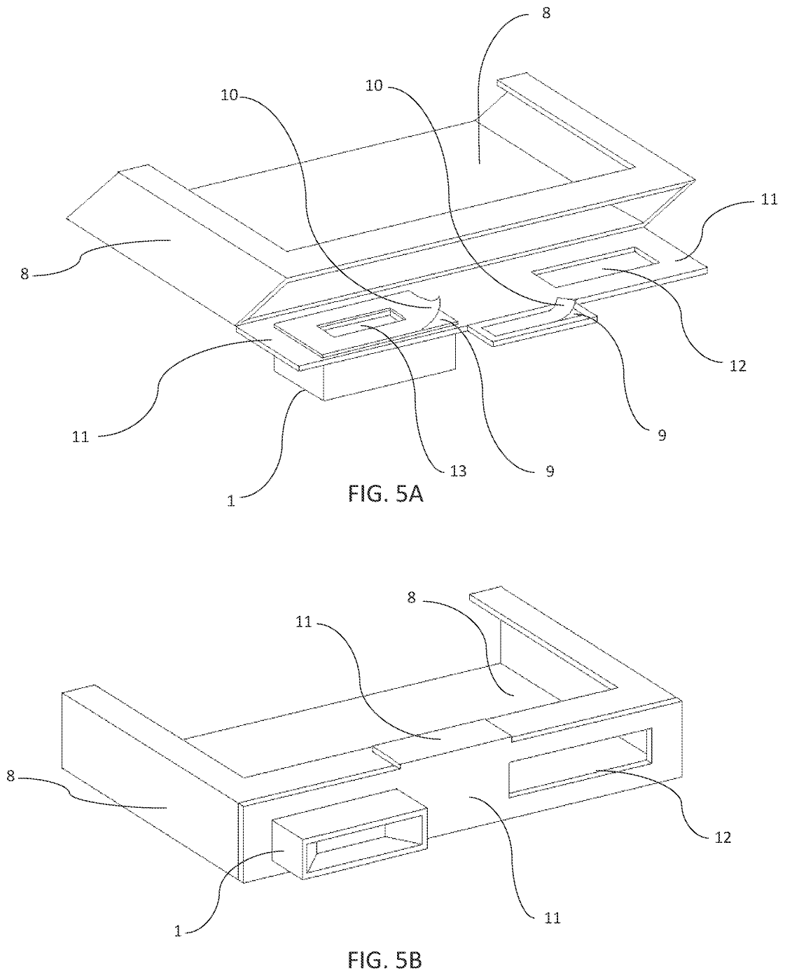

FIG. 5A introduces the perspective view of an electronic device case (8) in conjunction with the stethoscope (1), designed for seamless attachment to an electronic device (14). The stethoscope (1) rests atop the case (8), which is specifically molded to accommodate the base (6) of the electronic device (14), ensuring a snug and secure fit. The case (8) is equipped with an adhesive layer (9) protected by a removable protective layer (10), facilitating a straightforward, secure attachment process to the electronic device (14). Additionally, the case (8) incorporates a cover (11) outfitted with strategically placed speaker openings (12) and microphone openings (13), aligned to ensure unimpeded sound transmission from the stethoscope (1) to the electronic device's microphone (16).

In FIG. 5B, the arrangement of the stethoscope (1) in relation to the case (8) is further clarified. The case (8), adorned with adhesive surfaces (9), is positioned to align with the microphone (16) of the electronic device (14). This strategic placement, aided by the adhesive bond, ensures optimal sound wave transmission from the stethoscope (1) directly to the electronic device. The cover (11) also features variable configurations for the speaker area (15) of the electronic device (14), allowing for either open or closed access depending on the specific requirements for sound isolation or ambient noise reduction.

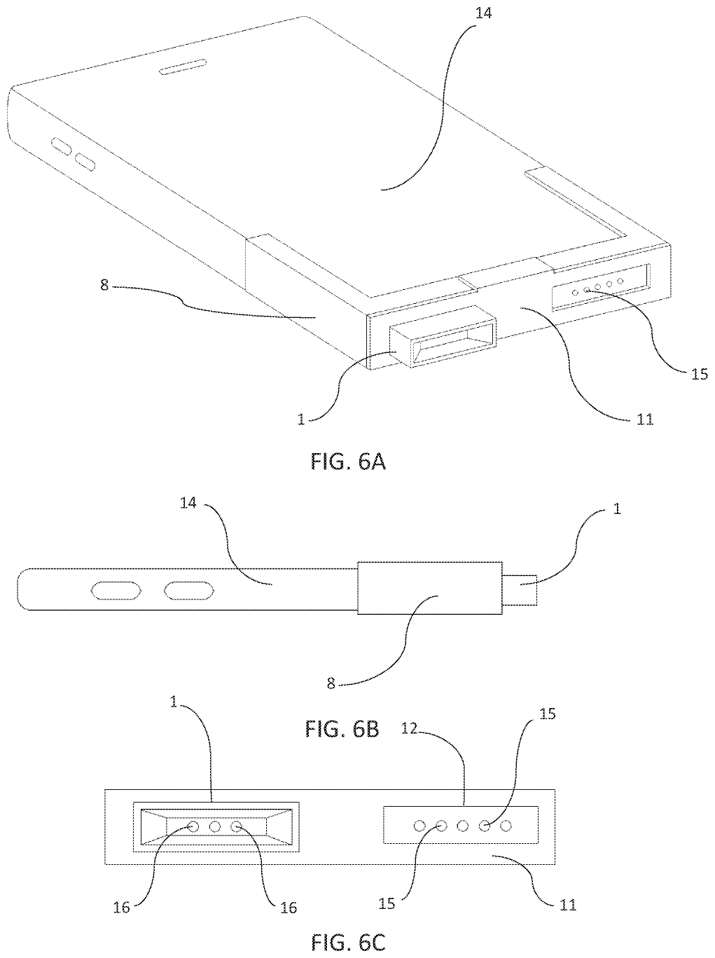

FIG. 6A illustrates the perspective view of the telemedicine stethoscope device (1) integrated with the case (8) and mounted on an electronic device (14). The case (8) is adeptly positioned on the electronic device (14), utilizing adhesive components (9) to secure the arrangement in place. This configuration ensures that the stethoscope (1) is optimally aligned with the electronic device's microphone (16), while also allowing for selective exposure of the device's surface areas. Notably, the design provides for the flexibility to either expose or conceal the speaker area (15) of the electronic device (14), depending on the user's preference or the specific requirements of the telemedicine session.

In FIG. 6B, the side view of this assembly is depicted, highlighting the strategic placement of the case (8) and the stethoscope (1) in relation to the microphone (16) of the electronic device (14). The adhesive surfaces (9) play a crucial role in firmly attaching the stethoscope (1) to the electronic device (14), ensuring a stable and efficient conduit for sound transmission directly into the microphone (16).

FIG. 6C provides a bottom view of the setup, showcasing how the stethoscope (1) encompasses the microphone (16) area of the electronic device (14). This enclosure can be achieved through a singular, unified structure of the stethoscope (1) or through a design incorporating multiple openings, catering to various microphone configurations. The cover (11), equipped with speaker openings (15), offers the option to be fully open or sealed, allowing for customization based on the need for sound isolation. This feature is particularly beneficial in scenarios requiring heightened audio clarity, where an adhesive layer may be employed to isolate the microphone (16) area completely, ensuring that sound waves are transmitted with minimal interference from external noise sources.

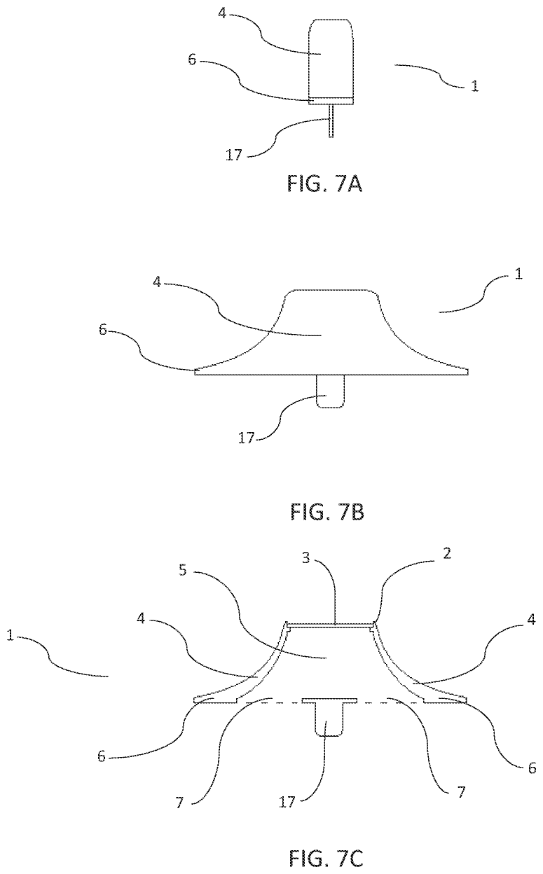

FIG. 7A presents a side view of an alternative configuration of the telemedicine stethoscope device (1), which incorporates a unique connection part featuring a protrusion (17). This protrusion (17) is specifically designed to insert into an opening of the electronic device (14), such as a charging port, to facilitate precise alignment of the stethoscope (1) with the electronic device's microphone (16). This ensures that sound is accurately captured and transmitted to the electronic device (14) for analysis during telemedicine consultations.

The design of the stethoscope (1) allows for the diaphragm (3) section to vary in width relative to the body (4), providing flexibility to accommodate different patient anatomies and auscultation needs. The protrusions (17), affixed to the base (6), play a critical role in securing the stethoscope (1) to the electronic device (14), with their placement being adjustable to match the specific layout of the electronic device's (14) ports and openings. This adaptability ensures that the stethoscope (1) can be used with a wide range of electronic devices (14), enhancing its utility in various telemedicine applications.

FIG. 7B offers a different perspective of the same stethoscope design (1), illustrating how the diaphragm (3) and the protrusion (17) can align. This configuration highlights the variable width of the body (4) as it extends towards the base (6), with the section housing the diaphragm (3) being narrower to focus the collection of sound waves. The strategic placement of the protrusion (17) on the base (6) ensures versatile attachment capabilities, catering to different electronic device (14) designs.

In FIG. 7C, a sectional view derived from FIG. 7B, the arrangement of microphone openings (7) around the protrusion (17) is detailed. These openings (7) are designed to encompass the microphone (16) of the electronic device (14), regardless of its specific location or design, thus ensuring that sound captured by the diaphragm (3) is efficiently transmitted. The presence of multiple microphone openings (7) allows for sound to be directed through various pathways within the body (4) of the stethoscope (1), accommodating devices where the microphone (16) and speaker (15) may be positioned differently.

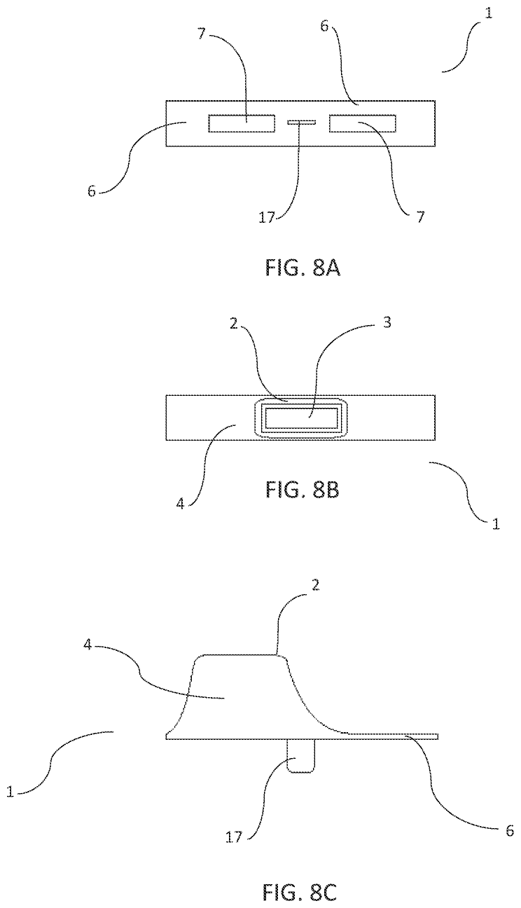

FIG. 8A provides a detailed bottom view of the telemedicine stethoscope device (1), showcasing the flexibility in the design of the microphone openings (7). These openings (7) are crucial for the transmission of sound from the stethoscope (1) to the electronic device (14) and can vary in number, ranging from a single aperture to a multi-part configuration. Their design is equally versatile, with options for straight polygonal, round, curved, or asymmetrical edges to best capture and direct sound waves. Accompanying these openings are protrusions (17), engineered to integrate seamlessly with any indentation or port on the electronic device (14), such as a charging port. The adaptability in the size and shape of these protrusions (17) ensures a secure fit and proper alignment of the stethoscope (1) with the electronic device (14), regardless of the device's make or model. Additionally, an adhesive layer covers the contact surface (6) of the stethoscope (1), excluding the areas of the microphone openings (7) and protrusions (17), to facilitate a stable attachment without impeding sound transmission.

In FIG. 8B, the top view of the stethoscope (1) is displayed, illustrating the positioning of the diaphragm (3) at the heart of the device. The placement of the diaphragm (3) can be either symmetrical, aligning directly with the central axis of the stethoscope (1), or asymmetric, adjusted to enhance sound collection efficiency based on specific user requirements or anatomical considerations. The shape of the diaphragm (3) mirrors this flexibility, with options for straight polygonal, round, curved, or asymmetrical designs, enabling precise customization to optimize acoustic performance.

FIG. 8C offers a side view of the stethoscope (1), emphasizing the potential for asymmetry in the construction of the body (4) and the base (6), as well as between the diaphragm (3) and protrusion (17). This design choice allows for tailored sound wave transmission pathways, ensuring that audio captured by the diaphragm (3) is effectively directed towards the microphone area (16) of the electronic device (14). The base (6) of the stethoscope (1) is designed to be elastic, accommodating electronic devices (14) of varying sizes by either fully or partially covering the microphone (16).

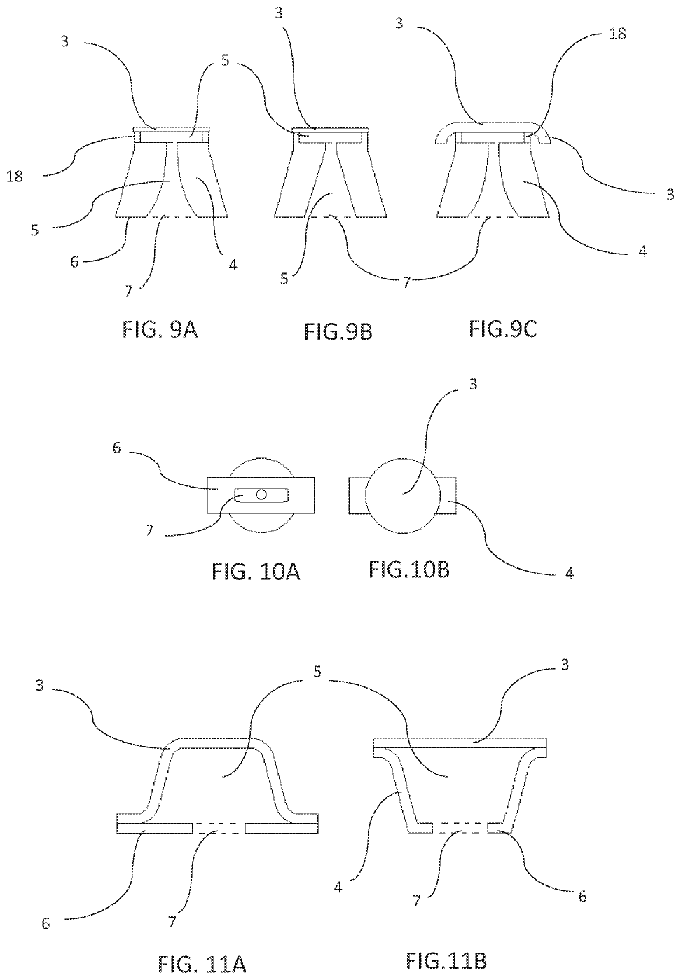

Between the base (6) and the diaphragm (3) of the telemedicine stethoscope device (1), the construction features side walls that delineate a pathway comprising one or more openings (5). These openings (5) are integral to the sound transmission process, with their inner surfaces designed to be either flat or contoured, adopting concave or convex shapes as demonstrated in FIGS. 9A and 9B. The variable width of these openings (5) from the diaphragm (3) towards the base (6) is a critical design element that may influence the movement of the diaphragm (3) by altering the sound wave path.

A specialized thin layer (18), as depicted in FIGS. 9A and 9C, is strategically placed between the sidewalls of the body (4) and the diaphragm (3). This layer (18) is crucial for adjusting the cross-sectional width of the opening (5), differing from the inherent thickness of the side walls. Composed of a flexible material such as plastic, foam, elastic rubber, or gel, layer (18) enables the diaphragm (3) to flex and move freely under applied pressure, thereby enhancing sound capture efficiency. Conversely, as shown in FIG. 9B, this layer (18) could also be crafted from a non-flexible material to maintain a consistent structure with the body (4), ensuring stability. Additionally, the edges of the diaphragm (3) are designed to be curved, as illustrated in FIG. 9C, to facilitate unobstructed movement essential for accurate sound transmission.

FIGS. 10A and 10B illustrate the versatility in the design of the diaphragm (3), which can adopt various shapes including round, square, rectangular, elliptical, or other irregular polygons with non-symmetrical edges or curves. This diversity in design allows the stethoscope (1) to be adapted for different operational needs and patient anatomies, improving the device's overall utility in telemedicine applications.

In another innovative arrangement showcased in FIG. 11A, the diaphragm (3) is seamlessly integrated with the stethoscope body (4), extending continuously to merge with the base (6). This continuous design not only simplifies the stethoscope's structure but also varies the thickness across different sections of the diaphragm (3), optimizing it for a range of sound frequencies. Some areas of the diaphragm (3) are thinner for enhanced sensitivity to higher frequency sounds, while thicker sections are better suited for capturing lower frequency sounds.

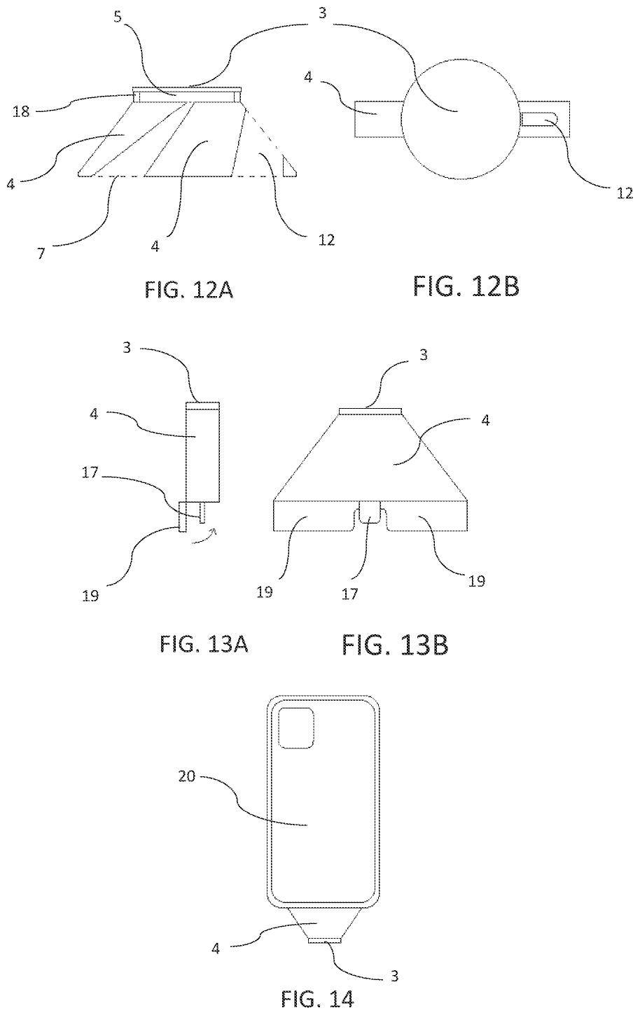

In FIG. 12A, the design of the telemedicine stethoscope device (1) is showcased with multiple openings (5) strategically positioned within the body (4) of the stethoscope. These openings (5), varying in number and location, serve as conduits for sound transmission, extending from the area between the base (6) and the diaphragm (3) to potentially including free openings directed towards the outer surface of the stethoscope (1). Notably, specific openings (12) are designed to align with external features of the electronic device (14), such as the speaker (15), facilitating optimal acoustic interaction and enhancing the device's utility in telemedicine applications by allowing sound to be captured or emitted directly through these targeted apertures.

FIGS. 13A and 13B detail the incorporation of a protective cover (19) on the base (6) of the stethoscope (1), engineered to safeguard the device's integral components and enhance its durability. This cover (19) is thoughtfully designed to adorn one or more edges of the base (6), featuring an adhesive layer for secure attachment and protection. It includes openings or mechanisms to accommodate protrusions (17), ensuring a seamless integration with the stethoscope body (4). In some designs, the protective cover (19) and the protrusion (17) are conceived as a unified element that, when activated, extends outward, promoting ease of attachment to the electronic device (14). This modularity allows the cover (19) to be customized for application across different areas of the stethoscope (1).

The stethoscope's base (6) is equipped with protrusions (17), either fixed or adjustable, designed to facilitate the secure and precise attachment of the stethoscope (1) to electronic devices (14) of varying dimensions. These protrusions (17) are characterized by their elasticity, allowing them to conform to the specific contours and ports of the electronic device (14).

FIG. 14 illustrates an innovative integration of the telemedicine stethoscope device (1) within an electronic device case (20), enhancing its functionality and utility. This design allows for the stethoscope (1) to either be an integral part of the case (20) or a detachable component that can be securely attached to the case (20) using various mechanisms such as protrusions, ridges, or magnetic elements. The versatility of the case (20) design means it can provide comprehensive coverage for the electronic device (14) or be tailored to protect specific areas.

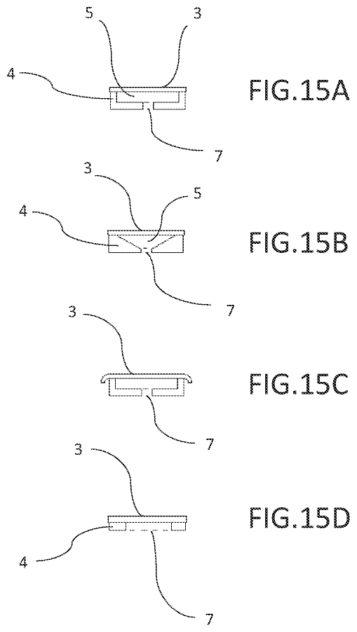

FIGS. 15A through 15D depict the stethoscope (1) in a compact form, focusing on the essential components of the diaphragm (3) and the body (4). This minimalist design emphasizes direct attachment of the diaphragm (3) to the body (4), allowing for a streamlined and efficient structure capable of assuming various shapes such as squares, rectangles, circles, polygons, or ellipses. The material selected for the body (4) is characterized by its elasticity and flexibility, incorporating magnetic properties in certain parts to facilitate attachment to metallic surfaces or elements within the electronic device (14). This adaptability ensures the stethoscope (1) can be easily positioned and repositioned for optimal sound collection, with the diaphragm (3) designed to be either flat or curved to suit different acoustic requirements.

The stethoscope (1) is described as being compatible with any electronic device (14) that includes a microphone, underscoring its broad applicability. Electronic devices (14) such as smartphones, smartwatches, tablets, Bluetooth microphones, and earbuds are all potential hosts for the stethoscope (1), which can be activated through simple positioning maneuvers. This activation process involves moving the stethoscope (1) to cover the device's microphone (16), thereby readying it for use in capturing and transmitting sound.

The telemedicine stethoscope device and its connecting parts are made from a variety of materials such as plastic, rubber, silicone, metal, and fabric, suitable for industrial manufacturing. It can be constructed from any single material or a combination, utilizing mold injection techniques for precise production.

Components including the skin contact piece, diaphragm, stethoscope body, electronic device connection points, and cases are produced either as individual parts or through multiple molds. They can be assembled together using chemical bonding or mechanical fastening, allowing for a modular design that can be tailored to specific needs.

Artificial intelligence (AI) enhances the device's functionality. It includes audio-quality classifiers to accurately identify heart, lung, and gastrointestinal sounds. A deep learning model assists users in correctly positioning the stethoscope and applying the right amount of pressure. Additionally, deep learning-based noise reduction improves the clarity of audio recordings. These features can be implemented alone or in combination, making the stethoscope adaptable for various telemedicine applications.

The entirety or each component comprising the stethoscope (1) can be produced from biodegradable, compostable, or recyclable materials. Biodegradable plastics encompass polylactic acid (PLA), polyhydroxyalkanoates (PHA), polybutylene adipate terephthalate (PBAT), polycaprolactone (PCL), starch-based plastics, and bioplastic PET (Bio-PET). These materials, which are not limited to those mentioned, include the following characteristics: These materials, derived from renewable resources or modified from traditional plastics, offer environmentally friendly alternatives that naturally break down over time, reducing pollution and waste accumulation. Compostable materials may originate from organic matter, cellulose, or paper, while recyclable materials may include paper, glass, or metal. The stethoscope can also be produced as a combination of various materials. Production methods may include one or more of the following steps: injection molding, cutting, assembling, welding.

The production can be carried out using the following methods: The continuous flow biodegradable or recyclable plastic production method comprises several interconnected steps. Initially, biodegradable polymer pellets or granules are fed into a mixing chamber where they are combined with additives and modifiers to achieve desired material properties. The mixture is then conveyed into an extrusion unit equipped with a twin-screw extruder. Within the extruder, the polymer blend is heated, melted, and homogenized under controlled temperature and pressure conditions. Following extrusion, the molten polymer is directed into a shaping die assembly designed to impart specific geometries to the plastic products. The die assembly may include interchangeable molds or nozzles to facilitate the production of various shapes and sizes. As the plastic material exits the die, it undergoes rapid cooling and solidification, preserving the desired form. To enhance the biodegradability of the final products, the manufacturing process can incorporate additives such as bio-based fillers, enzymes, or microbial agents that accelerate decomposition in natural environments. These additives are carefully integrated into the polymer blend during mixing to ensure uniform distribution and effectiveness. Additionally, the continuous flow nature of the production line enables high throughput and efficiency, minimizing downtime and waste. Automated monitoring and control systems oversee key process parameters, ensuring consistent product quality and performance. The disclosed continuous flow biodegradable plastic production method offers a sustainable approach to plastic manufacturing, utilizing renewable resources and environmentally friendly practices to produce biodegradable plastics suitable for a wide range of applications.

### CONCLUSION

Unless otherwise defined, all terms (including technical terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

The disclosed embodiments are illustrative, not restrictive. While specific configurations of the telemedicine stethoscope device of the invention have been described in a specific manner referring to the illustrated embodiments, it is understood that the present invention can be applied to a wide variety of solutions which fit within the scope and spirit of the claims. There are many alternative ways of implementing the invention.

It is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. A telemedicine stethoscope device comprising:
a skin contact element configured to establish a secure interface with a patient's skin, said skin contact element having a first opening;
a walled structure connecting said skin contact element to a base, said walled structure having an inner surface and an outer surface, wherein said inner surface defines a hollow space extending from the first opening to a second opening located in the base;
a detachable coupling mechanism incorporated into said base for removably attaching the device to an electronic device in such a manner that the second opening aligns with and securely encompasses a microphone of the electronic device;
a diaphragm membrane formed of a flexible material, said membrane positioned within the path defined between the first opening and the second opening, and arranged orthogonally to the direction of sound transmission between the first opening and the second opening, wherein the diaphragm membrane in conjunction with the hollow space forms a sound chamber for transmitting sound vibrations from the patient's skin through the device to the microphone of the electronic device;
whereby the device is adapted to facilitate the recording of heart, lung, and bowel sounds during video conference consultations when attached to the electronic device and placed against the skin of a patient.

2. The telemedicine stethoscope device of claim 1, wherein the skin contact element is further characterized by a layer of elastic and soft material that is adaptable to various skin surfaces, said material capable of temporarily adhering to the skin to maintain the position of the device during operation.

3. The telemedicine stethoscope device of claim 1, wherein the detachable coupling mechanism comprises an adhesive layer that enables secure attachment to and detachment from an electronic device.

4. The telemedicine stethoscope device of claim 1, wherein the detachable coupling mechanism comprises a casing that encapsulates the electronic device to create a sealed environment for enhanced sound transmission from the stethoscope to the electronic device's microphone.

5. The telemedicine stethoscope device of claim 1, wherein the detachable coupling mechanism comprises one or more protrusions designed for alignment with specific features on an electronic device, such as a charging port or audio jack.

6. The telemedicine stethoscope device of claim 1, wherein the diaphragm membrane is transparent or semi-transparent.

7. The telemedicine stethoscope device of claim 1, wherein the diaphragm membrane is opaque.

8. The telemedicine stethoscope device of claim 1, wherein the walled structure includes side walls that taper inward or outward, optimizing the acoustic properties of the sound chamber formed between the skin contact element and the electronic device's microphone.

9. The telemedicine stethoscope device of claim 1, wherein the device is constructed from materials selected from a group consisting of plastic, rubber, silicone, metal, fabric, biodegradable materials, and combinations thereof.

10. The telemedicine stethoscope device of claim 1, further characterized by the inclusion of a protective cover over the base, which may contain adhesive properties for secure placement, and an opening for accommodating protrusions or other alignment features.

11. The telemedicine stethoscope device of claim 1, further comprising multiple microphone openings located on the base to accommodate different models of electronic devices, ensuring universal compatibility and efficient sound transmission.

12. The telemedicine stethoscope device of claim 1, wherein the hollow space between the first and second openings is adjustable to modify the acoustics of the sound chamber, accommodating variations in patient anatomy and the specific auscultation requirements.

13. The telemedicine stethoscope device of claim 1, designed to be integrated with or attached to a variety of electronic devices including smartphones, tablets, and wearable technology, enabling versatile use in telemedicine applications.

14. The telemedicine stethoscope device of claim 1, incorporating artificial intelligence models designed to optimize audio-quality assessment for the detection and analysis of heart, lung, and gastrointestinal sounds, enhancing diagnostic capabilities.

15. A continuous flow biodegradable plastic production method comprising:
mixing biodegradable polymer materials with additives and modifiers in a mixing chamber;
extruding the polymer blend using a twin-screw extruder under controlled temperature and pressure conditions;
shaping the molten polymer into desired products using a shaping die assembly;
incorporating biodegradability-enhancing additives into the polymer blend during mixing to accelerate decomposition in natural environments;
automating monitoring and control systems to ensure consistent product quality and performance.
